(19) United States
(12) Patent Application Publication
(10) Pub. No.: US 2023/0326612 A1
(43) Pub. Date: Oct. 12, 2023

(54) Methods and Software for Contact Tracing and Exposure-Event Suppression Using Indoor Positioning

(71) Applicant: Everbridge, Inc., Burlington, MA (US)

(72) Inventors: Anahita Asrari, Waltham, MA (US); Imad Mouline, Braintree, MA (US)

(21) Appl. No.: 18/024,541

(22) PCT Filed: Sep. 13, 2021
(86) PCT No.: PCT/US2021/050073

§ 371 Date: Mar. 3, 2023

Related U.S. Application Data
(60) Provisional application No. 63/077,980, filed on Sep. 14, 2020.

(51) Int. Cl. G16H 50/80; H04W 4/02; H04W 4/12

(52) U.S. Cl. CPC G16H 50/80; H04W 4/023; H04W 4/025; H04W 4/12

(57) ABSTRACT

Methods and software for tracing contacts among people via exchanging of contact-tracing (CT) keys between mobile devices and treating various ones of the CT keys as infected CT keys after self-reporting events in which users have self-reported as having, at least potentially, been infected by a target infectious agent. In some embodiments, CT keys are exchanged between mobile devices when the mobile devices determine that they arm within a predetermined distance of one another and/or for a predetermined amount of time such that sufficient contact for infectious-agent transmission could have occurred. In some embodiments, mobile devices within the predetermined distance of one another can determine whether or not they are within a same separated space as one another (e.g., are not separated by a physical partition) as an additional level of intelligence to inform whether or not transmission of the infectious agent could have occurred.

### RELATED APPLICATION DATA

This application claims the benefit of priority of U.S. Provisional Patent Application Ser. No. 63/077,980, filed Sep. 14, 2020, and titled “METHODS AND SOFTWARE FOR CONTACT TRACING AND EXPOSURE-EVENT SUPPRESSION USING INDOOR POSITIONING”, which is incorporated by reference herein in its entirety.

### FIELD OF THE DISCLOSURE

The present disclosure generally relates to the field of contact tracing. In particular, the present disclosure is directed to methods and software for contact tracing and exposure-event suppression using indoor positioning.

### BACKGROUND

Identifying when two people come into close enough proximity to one another, and perhaps also for a long enough period of time, for an infection to spread from one of those two people to the other can have a meaningful impact on inhibiting or controlling the spread of an infectious disease. For example, knowing when a first person that has tested positive for the SARS-CoV-2 virus has been in “transmissive contact” with a second person, including in the recent past, can allow the second person, and any other person or people that the second person has had similar contact with, to be notified that they may have been infected. Consequently, all of the notified people can take appropriate action, such as self-quarantining and/or getting tested for the infection, to inhibit them from further spreading the infectious disease. Here, the term “transmissive contact” does not necessarily mean physical contact. Rather, “transmissive contact” in this context includes any physical proximity (distance) and/or time that have been established as parameters for which an infectious agent, such as a virus or flu, can be transmitted from one person to another. In one example for the SARS-CoV-2 virus, it has been proffered that the virus can be transmitted from one person to another if the two people are within 6 feet of one another for 15 minutes or more.

In the recent COVID-19 pandemic, contact-tracing apps for personal mobile smartphones have been developed and deployed for determining whether or not two people—as measured between the respective smartphones they are carrying—have been within 6 feet of one another for at least 15 minutes. Conventional smartphone contact-tracing apps typically use the BLUETOOTH® radios onboard the smartphones and the perceived signal strengths of the corresponding radio signals to estimate the relevant distance and time to determine whether or not sufficient transmissive contact has occurred for an infectious agent—if present—to be transmitted between the two people.

While these contract-tracing apps can provide significant benefits, they can, and do, provide false positives. For example, conventional contact-tracing apps will identify that two people have had sufficient transmissive contact for an infection to occur when they are, in fact, located in two separate and distinct spaces separated by a sealing partition, such as a sheetrock wall or glass wall. In such cases, if the two people were never in the same room together, there was virtually no chance of infection, despite them being closer together than 6 feet for 15 minutes or more. False positives can cause undesirable stress to the person or people that have been falsely identified as having been exposed to the infectious agent.

### SUMMARY

In one implementation, the present disclosure is directed to a method of notifying a first person that the first person has been in transmissive contact with a second person that has been infected with an infectious agent, wherein each of the first and second persons carries, respectively, a first mobile device and a second mobile device each containing contact-tracing (CT) software. The method being performed by the first mobile device includes receiving a first CT key transmitted by the second mobile device; receiving an infected CT key indicating that the second person has been infected by the infectious agent; matching the infected CT key and the first CT key to one another; and based on the matching, causing the first mobile device to notify the first person of the transmissive contact.

In another implementation, the present disclosure is directed to a method of controlling a first mobile device deployed in a contact-tracing (CT) system that employs a server for facilitating the CT system and that includes a second mobile device, wherein the first mobile device is carried by a first person and the second device is carried by a second person. The method being performed by the first mobile device includes continually generating, serially, first-mobile-device CT keys and storing the first-mobile-device CT keys; receiving from the first person a self-reporting of infection by an infectious agent; and based on the receiving of the self-reporting, transmitting a set of the first-mobile-device CT keys to the server.

In yet another implementation, the present disclosure is directed to a method of determining whether transmissive contact has occurred between two people carrying, respectively, first and second mobile devices. The method includes exchanging, between the first and second mobile devices, unique contact-tracing (CT) keys and location information in response to detecting that the first and second mobile devices are within a predefined transmissive proximity to one another; wherein each of the first and second mobile devices accepts or rejects the unique CT key received from the other of the first and second mobile devices based on whether or not the location information indicates that the first and second mobile devices are in a same separated space as one another.

### BRIEF DESCRIPTION OF THE DRAWINGS

For the purpose of illustration, the drawings show aspects of one or more embodiments of the invention(s). However, it should be understood that the present disclosure is not limited to the precise arrangements and instrumentalities shown in the drawings, wherein:

FIG. 1A is a diagram of a scenario involving an example contact-tracing (CT) system of the present disclosure and in which the CT system determines transmissive contact has occurred between two members of an organization located in proximity to one another;

FIG. 1B is a high-level block diagram of a mobile device implementing example CT software in accordance with the present disclosure;

FIG. 2 is a diagram of a scenario involving an example CT system of the present disclosure that includes location information and in which the CT system determines transmissive contact has occurred between two members of an organization located in proximity to one another in the same space;

FIG. 3 is a diagram of a scenario involving an example CT system of the present disclosure that includes location information and in which the CT system determines transmissive contact has not occurred between two members of an organization located in proximity to one another but in separated spaces;

FIG. 4 is a diagram of a scenario involving an example CT system of the present disclosure that includes location information and in which the CT system initially determines transmissive contact has not occurred between two members of an organization located in proximity to one another but in separated spaces and later determines that transmissive contact has occurred after one of the members enters the same space as the other member;

FIG. 5 is a flow diagram illustrating an example method of notifying a person that they have been in transmissive contact with another person that has self-reported as having been infected by an infectious agent;

FIG. 6 is a flow diagram illustrating an example method of controlling a mobile device deployed in a CT system;

FIG. 7 is a flow diagram illustrating an example method of determining whether transmissive contact has occurred between two people carrying corresponding respective mobile devices; and

FIG. 8 is a high-level schematic diagram/high-level block diagram illustrating an example implementation of a CT system of the present disclosure.

### DETAILED DESCRIPTION

In some aspects, the present disclosure is directed to contact-tracing (CT) systems and methods for anonymously identifying when people have come into transmissive contact with one another with a goal of tracing such contact in the event that any of the people report that they have been infected with an infectious agent, such as the current SARS-COV-2 virus, or any other relevant infectious agent identified now or in the future. This allows for alerting all of the people having contact chains that can be traced back to the infected person that they may have been infected with the infectious agent.

Before proceeding with describing example CT systems and other features and aspects of the present disclosure, it is noted those skilled in the art will understand that the examples provided herein are simplistic for ease of illustrating basic functionalities of the disclosed embodiments. Actual deployments would typically include many more organization members, many more deployments of mobile CT software, and more complex and numerous transmissive contacts among the monitored members. Those skilled in the art will readily understand how to design and deploy CT systems of any size and in any physical setting(s) without undue experimentation using the present disclosure as a guide.

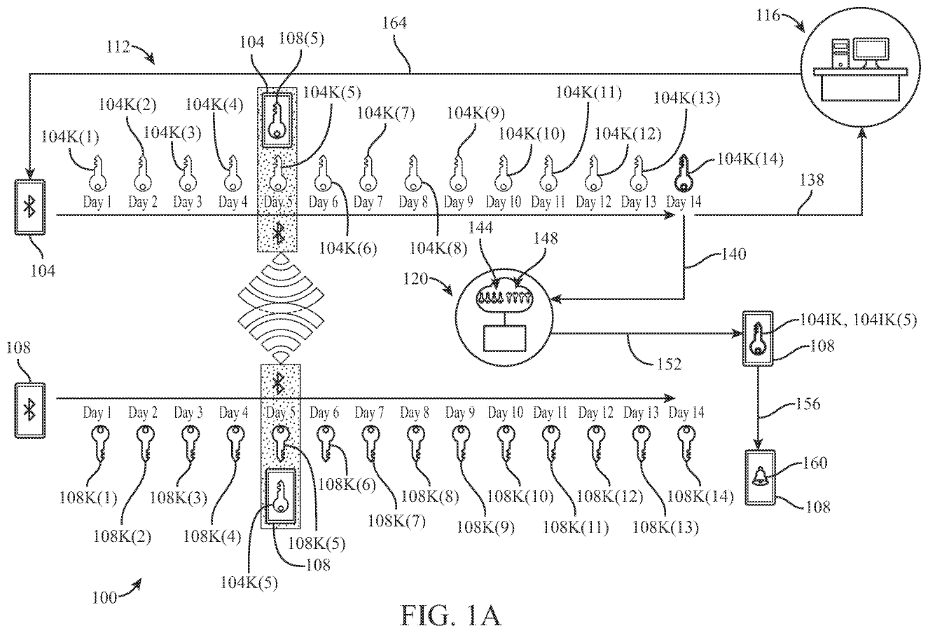

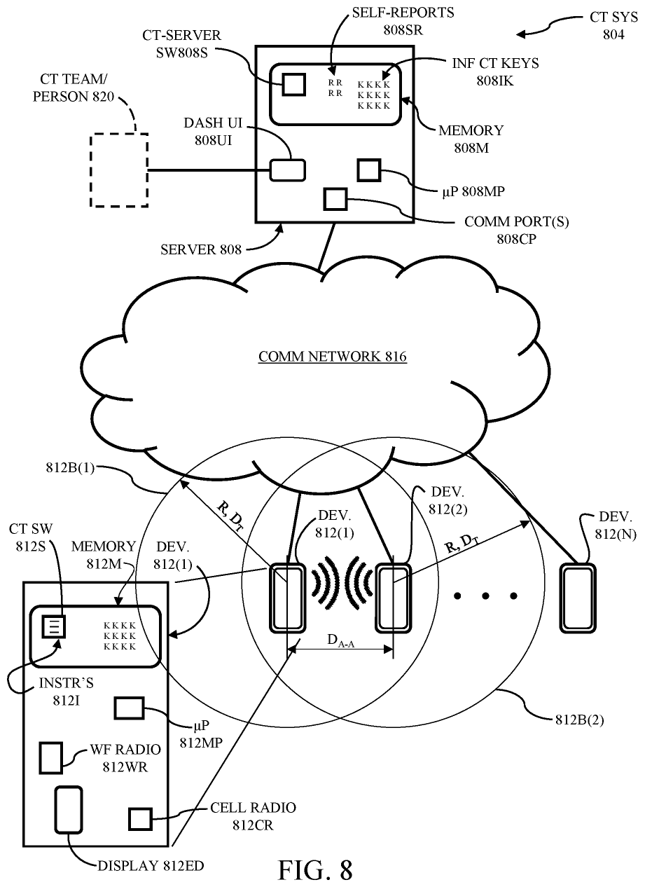

Referring to FIG. 1A and also occasionally to FIG. 1B, FIG. 1A illustrates an example scenario 100 of proximity-based contact tracing performed in accordance with the present disclosure. In this example, each of two people, “Emily” and “Eric” for convenience, carriers, or otherwise keeps in close proximity (e.g., within arm's reach), a corresponding mobile device 104, 108 which may be, for example, a cellular smartphone that includes, among other things, a radio 104R, 108R (see also FIG. 1B), such as a BLUETOOTH® low energy (BLE) radio, and contains CT software 104S, 108S (FIG. 1B), such as, for example, a dedicated CT mobile app, customized to provide the requisite functionalities, including the functionalities described herein, among others. Referring to FIG. 1B, each mobile device 104 and 108 further includes memory 104M and 108M, one or more microprocessors 104MP and 108MP, and human-machine interface (HMI) 104HMI and 108HMI, which may include any one or more types of interfaces that allow a user to interact with the mobile device, most commonly, but not limited to, a touchscreen. Details of an example mobile device suitable for use as either of the mobile devices 104 and 108 are described below in connection with the mobile devices 812(1)-(N) of FIG. 8. Those skilled in the art will readily understand that the mobile devices 104 and 108 need not be smartphones and that the radios 104R and 108R may be any suitable type of radio or replaced with one or more other wireless communication devices, such as optical or ultrasonic communications devices, among others.

In one scenario, the CT software 104S and 108S on Emily's and Eric's mobile devices 104 and 108 may be part of an overall CT system 112 deployed by an organization, such as a company, educational institution, government, etc., for anonymously monitoring interactions of organization members (Emily and Eric in this example) with one another with the goal of making the members of the organization as safe as possible from becoming infected by one or more infectious agents, such as a virus or flu, among others. An important feature of embodiments of CT systems disclosed herein, such as the CT system 112 of FIG. 1, is the anonymity of the members. Typically and generally, the identities of infected members are, at most, known to members of a specially designated CT team or person, such as the CT team/person 116 of the CT system 112 of FIG. 1. Such a CT team/person may be part of, for example, a human-resources department of the relevant organization.

In the example of FIGS. 1A and 1B, the CT software 104S and 108S aboard the mobile devices 104 and 108 uses the corresponding respective radios 104R and 108R aboard those mobile devices to determine proximity of the mobile devices and, therefore, Emily and Eric, to one another, as well as to transmit anonymous and unique CT keys, such as CT keys 104K(1) to 104K(14) and 108K(1) to 108K(14) in this example scenario 100, to one another based on sufficient proximity determination, as discussed below in detail. In some embodiments, tracking can be minimized and anonymity can be maximized by continually generating new CT keys, such as CT keys 104K(1) to 104K(14). Also in this example, the CT software 104S, 108S on each mobile device 104, 108 generates a new CT key (e.g., a seed key) every 24 hours, here one on each of the daily CT keys 104K(1) to 104K(14) and 108K(1) to 108K(14) over a period of 14 days. In a nonlimiting example, each CT key is 32 bytes long and is generated using any suitable algorithm, such as a hash-based message authentication code (HMAC) algorithm, for example, an HMACSHA256 algorithm. One skilled in the art will readily understand that each CT key, including the CT keys 104K(1) to 104K(14) and 108K(1) to 108K(14) shown in FIG. 1A, may have any suitable format and be generated using any suitable key-generation algorithm. In some embodiments, when a CT key is uploaded as part of a self-report (e.g., when Emily or Eric self-reports as being infected with the relevant infectious agent (e.g., the SARS-CoV-2 virus) on Day 14 (see below)), it is serialized as a hexadecimal string, which in this example will be 64 characters long, for storage on a server 120, which may be, for example, a decentralized server for robustness.

FIG. 1A shows in this example that, on Day 5, Emily's and Eric's mobile devices 104 and 108 (and Emily and Eric) come into transmissive contact one another as determined by the relevant transmissive-contact parameter(s), such as distance (proximity) and/or time. In one example relative to the SARS-CoV-2 virus, the transmissive-contact distance and time are “less than 6 feet” and “15 minutes or more”, respectively. Because Emily's and Eric's CT software 104S and 108S have determined transmissive contact by proximity, the CT software on Emily's and Eric's mobile devices 104 and 108 exchange their current, i.e., Day 5, CT keys 104K(5) and 108K(5) with one another via their respective radios 104R and 108R, and the respective CT software stores the received CT keys in the memories 104M and 108M aboard the corresponding respective mobile devices, as illustrated in FIG. 1A by the representations of the CT keys 104K(5) and 108K(5) in the screen regions of the mobile devices. In this example scenario 100, Day 5 was the only day that Emily and Eric were in transmissive contact with one another as determined by Emily's and Eric's CT software 104S and 108S.

In this example scenario 100, on Day 14 Emily self-reports, as illustrated by arrow 138, that she has been infected with the relevant infectious agent. For example, Emily may have tested positive for the infectious agent and/or presented with symptoms of the infectious agent. In this connection and in an example, the CT software 104S and 108S aboard each of the mobile devices 104 and 108 may implement a self-reporting feature 124 (FIG. 1B), such as a self-reporting soft button or other soft selector that initiates a self-reporting algorithm 104SR and 108SR (FIG. 1B) of the CT software 104S and 108S. As part of the self-reporting, Emily may optionally be prompted to provide various information, for example, via a fillable form 132 (FIG. 1B) displayed on Emily's mobile device 104 by the self-reporting algorithm, about the determination of the infection, perhaps along with the phone number of her mobile device and/or other relevant identification information. The CT software 104S and 108S may provide a send-report feature 136 (FIG. 1B), such as a soft button or other control, that, when Emily selects it, causes her CT software 104S to send the information she input, along with other information, including all of Emily's stored anonymous CT keys (as illustrated by arrow 140, to the server 120 (e.g., an enterprise server or decentralized cloud server) that is tasked with taking actions in response to receiving Emily's self-reporting event. As seen in the cloud portion of the server 120, the server stores received non-infected CT keys (not illustrated), received potentially infected CT keys (collectively, at 144), and received infected CT keys (collectively, at 148), including all of Emily's reported CT keys 104K(1) to 104K(14) in this example (note that Emily's CT keys are not identified separately among CT keys 144 and 148 stored on the server 120 to avoid cluttering the figure). In this example, Emily's CT key 104K(14) from the day she reported as being infected may be considered an infected CT key and any of Emily's other CT keys, here, CT keys 104K(1) to 104(13) may be either a non-infected CT or a potentially infected CT key, depending on factors such as how long Emily experienced symptoms prior to getting tested, how long it took for Emily to receive the test results, and the incubation period of the infectious agent, among others. For the sake of this disclosure and the appended claims, potentially infected CT keys are identified as infected CT keys, since anyone in contact with Emily during a time when she may have been infected would typically follow the same protocols they would follow had they been in contact with her when she was actually infected. Here, all of Emily's CT keys 104K(1) to 104K(14) are identified as infected CT keys.

In this example scenario 100, as illustrated by arrow 152, each mobile device that is part of the CT system 112, including Eric's mobile device 108, polls the server 120 to retrieve, or pull, any infected CT keys that the server has stored since the last time that device polled the server. In this example, Emily's CT keys 104K(1) to 104K(14) are included in such new infected CT keys. The stored CT keys that Eric's mobile device 108 received from one or more other mobile devices of the overall CT system 112 during a transmissive-contact key exchange includes Emily's CT key 104K(5). Therefore, when Eric's mobile device 108 receives Emily's infected CT keys (collectively represented at 1041K in FIG. 1A) from the pull, Eric's CT software 108S will compare data from Emily's infected CT keys with data from each of Eric's stored CT keys 108K(1) to 108K(2) to anonymously determine whether or not any of the received infected CT keys match any of Eric's stored received CT keys from one or more other mobile devices, and will find a match between Emily's infected Day 5 CT key 104IK(5) received from the server 120 and Emily's Day 5 CT key 104K(5) received and stored on the day of the transmissive contact between Emily and Eric, i.e., Day 5. It is noted that while the present implementation involves each mobile device 104, 108 pulling infected CT keys, here including CT keys 104K(1) to 104K(14), from the server 120, in other implementations, the server may push infected CT keys out to the mobile devices.

As indicated by arrow 156, this match signals to Eric's CT software 108S that there is a chance that Eric was infected by the transmissive contact he had with Emily on Day 5. In turn, Eric's CT software displays an exposure notification 160 on Eric's mobile device 108 that notifies Eric that he may have been infected and should take appropriate response measure(s), such as getting tested for the infectious agent and self-reporting if positive, and/or self-quarantining, among other things. If Eric gets tested and the test is positive for the infectious agent, Eric should self-report so that the CT system 112 can then handle his newly infected CT keys (not shown) in the same manner as Emily's infected keys 104K(1) to 104K(14), including making them available to the mobile devices of those organization members that may have been in transmissive contact with Eric after Day 5, since those members may have become infected, too, from their transmissive contact with Eric.

On the CT team/person 116 side, a member of the CT team knows that Emily self-reported, and thus and as indicated by arrow 164, he can follow up with her directly as needed to obtain more information from Emily and/or provide information to her. For example, John may know how to contact Emily—who is otherwise anonymous via the anonymous CT key process—by virtue of Emily providing her phone number and/or other contact information during the self-reporting discussed above. As noted above, the server 120 designated Emily's Day 5 CT key 104K(5) as an infected CT key 1041K on Day 14 in response to Emily self-reporting on Day 14. In some embodiments, the designation of infected CT keys might not happen automatically in response to an initial self-reporting. For example, the CT team/person 116 may contact the self-reporter to confirm the legitimacy of the self-reporting to ensure that the self-reporting was valid and not, for example, accidental or done maliciously by someone other than Emily gaining access to her mobile device 104. Therefore, in some embodiments, the server 120 may have a dashboard that provides the CT team/person 116 with control over the treatment of CT keys, perhaps among other functionality(ies).

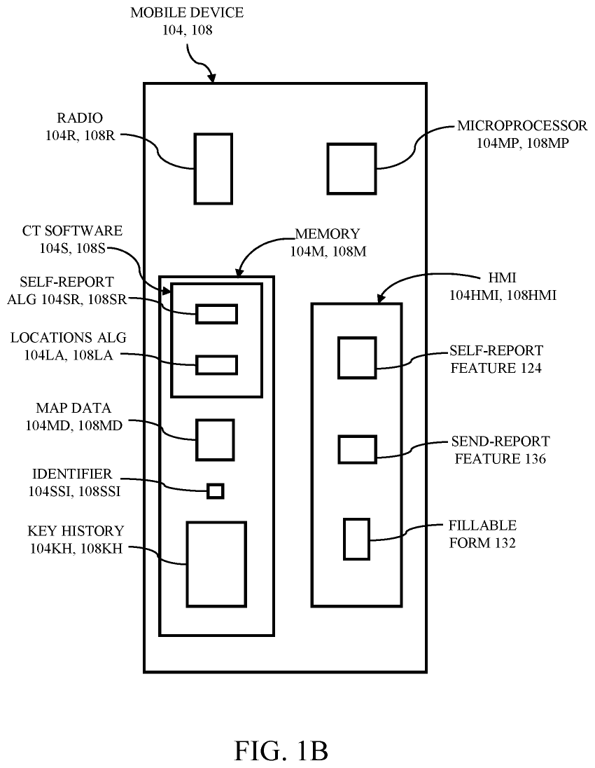

FIG. 2 illustrates a scenario 200 that includes a CT system 212 that may be similar to the CT system 112 of FIG. 1A but that is enhanced to consider location information for more accurate determinations of whether or not transmissive contact has occurred between any two or more members of an organization. For convenience and simplicity, the scenario 200 of FIG. 2 involves the same organization members, Emily and Eric, and utilizes the CT software 104S and 108S respectively aboard Emily's and Eric's mobile devices 104 and 108 (FIG. 1B) but enhanced with location algorithms 104LA and 108LA for obtaining and processing location information as discussed in detail below. It is noted that all unlabeled features, aspects, and components in FIG. 2 are the same as or similar to the corresponding features, aspects, and components labeled in FIG. 1A.

As discussed above relative to FIGS. 1A and 1B, the CT system 112 of FIGS. 1A and 1B uses the radios 104R and 108R aboard the mobile devices 104 and 108 to sense proximity of the mobile devices, and therefore, the organization members carrying the mobile devices (there, Emily and Eric), to one another. In the example enhanced CT system 212 of FIG. 2, the CT system also uses a set of beacons, such as the four BLE beacons 216(1) to 216(4) shown in a separated space 220 (e.g., room) on the left-hand side of FIG. 2, that allows the individual mobile devices (e.g., via their CT software 104S and 108S or other software) to triangulate their positions relative to the beacons. In some embodiments, when these locations are coordinated with a map of the relevant facility (not shown) of which the separated space 220 is a part, the CT system 212 can augment the proximity-based transmissive contact analysis with location metadata (e.g., a separated-space identifier) to eliminate certain types of false-positive transmissive contact determinations that using only proximity information will cause.

For example and as mentioned above, a false-positive can arise in a situation in which two mobile devices determine that they are within infection-transmission distance of one another, but where the mobile devices, and corresponding organization members, are effectively isolated from one another such that infection transmission cannot occur. Examples of such effective isolation include situations where a partition exists between the two organization members, such as in the case of the organization members being located in adjacent spaces, for example, rooms, physically isolated from one another by a sheetrock, glass, or other type of wall or partition, such that they are considered separated spaces. In these cases, the wall/partition between the two organization members provides a barrier to the transmission of the infecting agent.

In some embodiments, the mobile devices 104, 108 each include map data 104MD, 108MD, as shown on FIG. 1B, (e.g., representing a floor plan) of the relevant facility that relates the beacons, here, beacons 216(1) to 216(4), to individual separated spaces (e.g., rooms) within the facility. In this manner, when each mobile device 104, 108 triangulates its position, the corresponding CT software 104S, 108S uses the resulting location and map data to identify which separated space that particular mobile device is located in. Then, to determine whether or not the two mobile devices 104 and 108 are actually in a transmissive-contact situation, the CT system 212 compares the now-identified space(s) with one another, here via location metadata. If the CT system 212 determines that the two location metadata match, i.e., that both mobile devices 104 and 108 and, by implication, both Emily and Eric, are in the same separated space, then the transmissive-contact scenario that the proximity-based determination identified is confirmed or accepted. However, if the CT system 212 determines that the location metadata do not match, i.e., that both mobile devices 104 and 108 and, by implication, both Emily and Eric, are in separate spaces isolated from one another such that an infection cannot be transmitted, then the transmissive-contact scenario that the proximity-based determined identified is denied or rejected.

In some embodiments, the CT system 212 determines whether or not the two mobile devices 104 and 108 are located in the same separated space, such as separated space 220, via the CT software 104S and 108S running on the corresponding mobile devices. This may be accomplished by the CT software 104S and 108S on the two mobile devices 104 and 108, respectively, sending its location metadata, as determined in this example via the triangulation noted above, to the CT software on the other of the two mobile devices. Since the CT software 104S and 108S of each mobile device 104 and 108 has determined its own location, it can determine whether or not the corresponding two mobile devices are in the same space, such as space 220, by comparing the location metadata received from the other mobile device with its own location metadata.

FIG. 2 illustrates an example of this exchange of location metadata. In this example, the CT software 104S, 108S on each mobile device 104, 108 generates a unique CT key (here, a daily generated CT key, just like in the example scenario 100 of FIGS. 1A and 1B (see CT keys 204K(1) to 204K(14) and 208K(1) to 208K(14) in FIG. 2). Then, by virtue of the proximity of the two mobile devices, the CT software of each mobile device sends both the generated CT key (CT keys 204K(5) and 208K(5) in the scenario 200 of FIG. 2) and the location information of that mobile device as location metadata to the other of the mobile devices. In the example shown in FIG. 2, both Emily's and Eric's mobile devices 104 and 108 are located in the same separated space 220 (“Room A”). Therefore, when the corresponding CT software 104S, 108S determines that the location metadata of the two mobile devices 104 and 108 in the exchange match one another, the CT system 212 identifies that a transmissive contact has indeed occurred. In an example, the relevant CT software 104S, 108S can use the location-based confirmation of the proximity transmissive contact as a triggering event to store, on the corresponding mobile device 104, 108, the CT key (here, either CT key 204K(5) or 208K(5)) it received from the other mobile device so as to become part of the exchanged-key history stored on that mobile device. Other aspects of the CT system 212 of FIG. 2 based on key exchange and self-reporting may be the same or similar to the corresponding aspects of the CT system 112 of FIGS. 1A and 1B. It is noted that while this and other examples involving determination and use of location metadata use a localized beacon, alternative means can be used for determining the location information, such as the Global Positioning System (e.g. for uncovered facilities) or other positioning system.

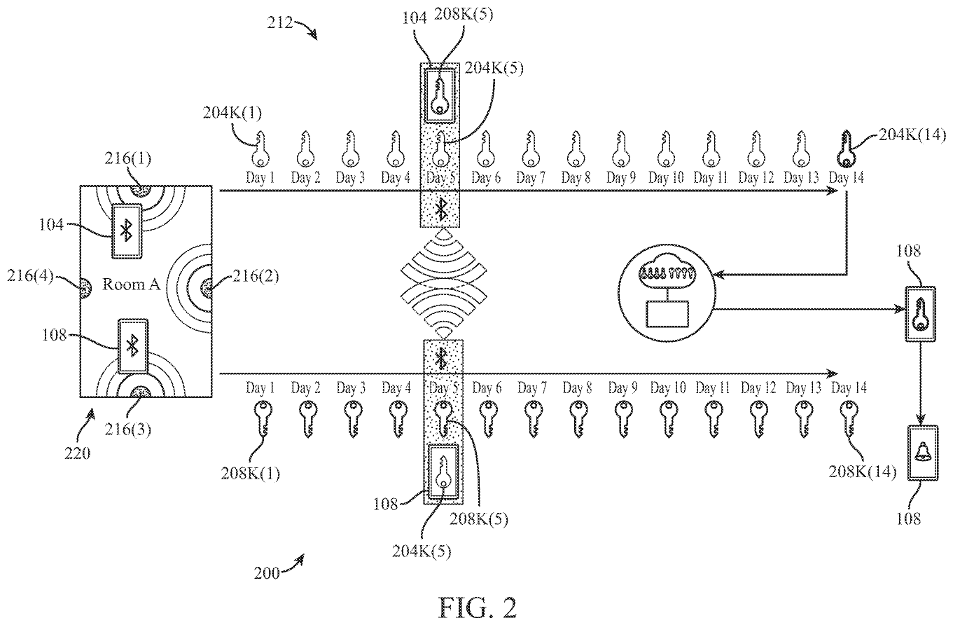

While FIG. 2 illustrates the exchange and subsequent acceptance of CT keys, here, CT keys 204K(5) and 208K(5) based on location-confirmed proximity-based transmissive contact, FIG. 3 illustrates an example scenario 300 in which the CT software 104S, 108S on each of the two mobile devices 104, 108 (FIG. 1B) rejects the CT key, here, the CT keys 204K(5) and 208K(5), received from the other mobile device because the location metadata (e.g., identified separated space) received from the other CT software does not match the location (e.g., identified separated space) in which the receiving mobile device is located. Referring to FIG. 3 and also to FIG. 1B, in the scenario 300 of FIG. 3, the proximity of Emily's mobile device 104 to Eric's mobile device 108 as determined by the CT software 104S and 108S and the strengths of the radio signals that the corresponding radios 104R and 108R receive indicates that a transmissive-contact may be occurring between Emily and Eric. In response to this determination, the CT software 104S, 108S of each of the two mobile devices 104, 108 sends its daily generated CT key, here CT keys 204K(5) and 208K(5), to the other mobile device.

However, the CT software 104S, 108S on each of the mobile devices 104, 108 also triangulates the position of the corresponding mobile device using beacons positioned throughout a facility 304, or a portion thereof, where monitoring of transmissive contacts is desired. In the example shown in FIG. 3, the facility at issue includes three separated spaces, namely, “Room B” 308, “Room C” 312, and “Room D” 316. In this example, each of the three separated spaces has at least two beacons located therein, with Rooms B 308 and C 312 each having three beacon, namely beacons 308(1) to 308(3) and 312(1) to 312(3), respectively and Room D 316 having two beacons, namely beacons 316(1) and 316(2). In this example, Rooms B through D 308, 312, and 316 are separated by partitions 320(1) to 320(3) that do not significantly block or degrade the strength of the signals from the beacons 308(1) to 308(3), 312(1) to 312(3), 316(1) and 316(2), so triangulation within Room D can be achieved, for example, using the two beacons 316(1) and 316(2) in Room D and one or both of the beacons 308(1) and 312(1) located in Rooms B and C along the partitions 320(2) and 320(3) separating Room D from corresponding respective Rooms B and C. In some implementations, a partition or other obstacle may degrade signal strength from the beacons 308(1) to 308(3), 312(1) to 312(3), 316(1) and 316(2), to a greater extent. However and as those skilled in the art will readily appreciate, the attenuation of one or more signals due to a partition or other obstacle can simplify the determination of which separated space, here Room B” 308, “Room C” 312, and “Room D” 316, that the relevant one(s) of the mobile devices 104 and 108 is/are in.

In the same manner as discussed above relative to FIG. 2, the CT software 104S, 108S on each of the mobile devices 104, 108 triangulates the position of the corresponding mobile device using effective ones of the beacons 308(1) to 308(3), 312(1) to 312(3), 316(1) and 316(2) and then uses that location and map data 104MD, 108MD to determine location information/metadata, such as a separated-space identifier 104SSI, 108SSI (here, one of “Room B”, “Room C”, and “Room D” 308, 312, 316), or other location information. Since Emily's and Eric's CT software 104S and 108S already identified their proximity of the mobile devices 104 and 108 to one another (perhaps along with a minimum time duration of maintaining that proximity), they will transmit their daily (in this example) unique CT keys, here CT keys 204K(5) and 208K(5), to the other of the two corresponding mobile devices. In addition to the software 104S, 108S on each of the mobile devices 104, 108 sending its CT key, such software will also transmit the separated-space identifier 104SSI, 108SSI to the other mobile device. The software 104S, 108S compares the received separated-space identifier 104SSI, 108SSI to its own location information to determine whether or not the two mobile devices 104 and 108, and, therefore, Emily and Eric, are located in the same separated space (here, room) as one another such that an infection could be transmitted and the proximity truly being a transmissive-contact situation.

In one example, Emily's CT software 104S sends “Room B” as the separated-space identifier 104SSI, and Eric's CT software 108 sends “Room C” as the separated-space identifier 108SSI via the BLE radios 104R and 108R, respectively. As illustrated in FIG. 3, when Emily's mobile device 104, which her CT software 104S has identified as being located in Room B, receives Eric's “Room C” separated-space identifier 108SSI then Emily's CT software compares the two separated-space identifiers and determines that they are not the same and that, therefore, Emily and Eric are not in the same room (space). Consequently, each of Emily's and Eric's CT software 104S, 108S determines that the proximity-determined contact is not an actual transmissive contact. In response and in one example, each of Emily's and Eric's CT software 104S, 108S does not store (or in other words, rejects), respectively, Eric's Day 5 CT key 208K(5) and Emily's Day 5 CT key 204K(5) in the respective exchanged-key history 104KH, 108KH stored in the memories 104M and 108M of Emily's and Eric's mobile devices 104 and 108. Consequently, when Emily self-reports on Day 14 and her CT software 104S sends the infected CT keys (collectively represented at 204IK), as illustrated by arrow 324, to the server 120 and pulls Emily's infected CT keys from the server as illustrated by arrow 328, Emily's Day 5 CT key 204K(5) will not be among the CT keys stored on Eric's mobile device because it was rejected in the transmissive-contact determination. Therefore, Eric's mobile device 108 will not find a match for Emily's infected Day 5 infected CT key 204IK(5) and Eric's mobile device 108 will not notify Eric, as illustrated at 332. It is noted that all unlabeled features, aspects, and components in FIG. 3 are the same as or similar to the corresponding labeled features, aspects, and components in FIGS. 1A and 2.

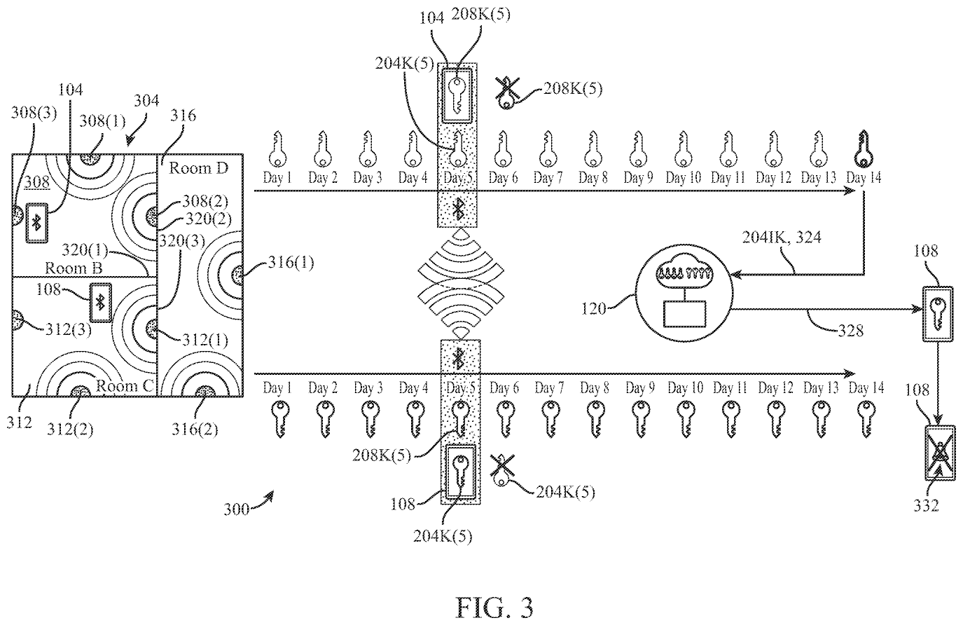

FIG. 4 illustrates a scenario 400 in which Emily was initially in a different separated space than Eric as per FIG. 3 (Emily was in Room B 308 isolated from Eric in Room C 312) such that after Emily's and Eric's CT software 104S and 108S exchanged their unique CT keys 204K(5) and 208K(5), neither of the CT apps stored those keys because an infection could not be transmitted due to the presence of a physical barrier, here partition 320(1) between Rooms B 308 and C 312. However, at some point after that initial non-transmissive-contact determination as per FIG. 3, Emily moves into Room C 312 so that she is now in the same separated space as Eric such that a transmissive-contact can occur.

In the scenario 400 illustrated in FIG. 4, and referring to FIG. 1B, each of Emily's and Eric's CT software 104S, 108S (FIG. 1B) generates a corresponding unique CT key, here CT keys 404K(1) to 404K(14) and 408K(1) to 408K(14), respectively, more frequently than every day, such as every 10 minutes. In some embodiments, not only does the more-frequent CT key generation assist with determining whether or not Emily and Eric are in the same separated space, but it also minimizes location tracking. Based on these new unique CT keys 404K(1) to 404K(14) and 408K(1) to 408K(14), the CT software 104S, 108S on each of the two mobile devices 104′, 108S′ may now interact with one another again based on their proximity to one another. In other words, the new unique CT keys 404K(1) to 404K(14) and 408K(1) to 408K(14) may restart the process of interacting with CT software 104S, 108S previously interacted with, such that the locations of the corresponding mobile devices 104 and 108 may be reassessed on the chance that one, the other, or both locations have changed such that the two devices are now in the same separated space. In some embodiments, each mobile device 104, 108 may not generate a new CT key on a set schedule. For example, in some embodiments, each mobile device 104, 108 may generate a new CT key based on change in location. In some embodiments, the change in location may be a move from one separated space to another, for example, one of Rooms B to D 308, 312, 316. In some embodiments, the change in location may be based on moving a certain distance and/or direction, among other things. In some embodiments, each mobile device 104, 108 may generate a new CT key on a set interval or other schedule, such as every 5 minutes, 10 minutes, etc., and, if the mobile device is moved according to requisite criteria between such intervals or scheduled generation, the mobile device may also generate a new CT key based on the transition from one separated space to another.

Referring still to FIG. 4, in the example shown Emily's location does indeed change from Room B 308 to Room C 312 where Eric is still present. In an example, this change in location of Emily's mobile device 104 may cause Emily's CT software 104S to generate a new unique CT key. In this case, the new unique CT keys allow the proximity- and location-determination process to start again. This time, when the proximity of Emily's and Eric's mobile devices 104 and 108 are within a predetermined transmissive contact range of one another when Emily's mobile device is now in Room C 312, the CT software 104S and 108S on both mobile devices determines their respective location to be Room C. Thus, Emily's and Eric's CT software 104S and 108S each transmit the same separated-space identifier 104SSI, 108SSI (here, “Room C”) to the other CT software and the follow-on separated-space-identifier comparison step determines that the two mobile devices 104 and 108 are in the same room. As a result, the CT software 104S and 108S store the received CT keys 404K(5) and 408K(5), i.e., Emily's CT software 104S stores Eric's received CT key 408K(5), and Eric's CT software 108S stores Emily's received CT key 404K(5), because now that Emily and Eric are in the same room, i.e., Room C 312, the contact could actually result in transmission of an infection should either of Emily or Eric be infectious and therefore would be identified as a transmissive contact. In the example illustrated, just as in FIGS. 1A and 2, when Emily self-reports that she is infected with the infectious agent, Eric will be rightly notified of exposure from the transmissive contact with Emily. It is noted that all unlabeled features, aspects, and components in FIG. 4 are the same as or similar to the corresponding labeled features, aspects, and components in FIGS. 1A, 2, and 3.

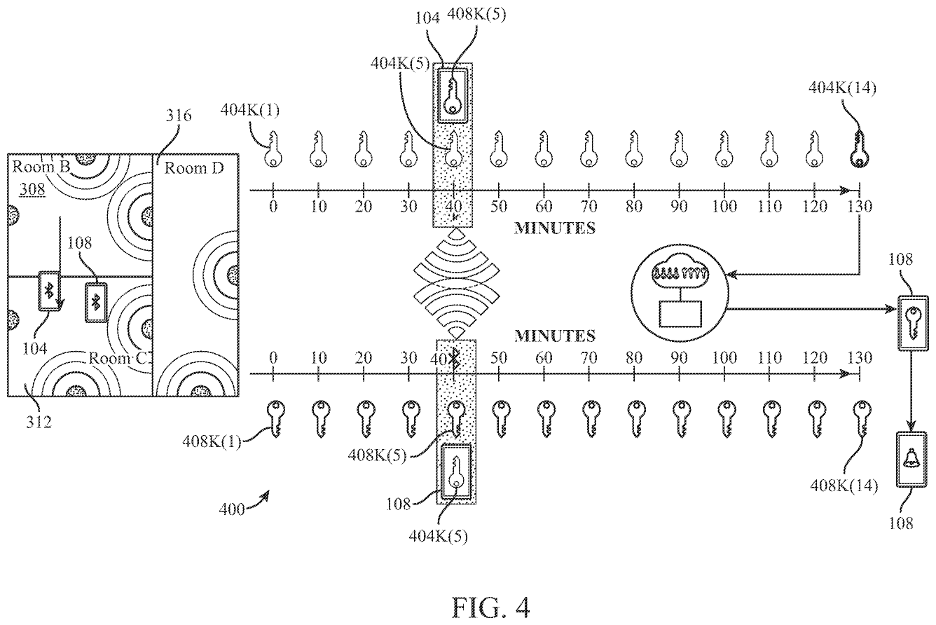

With reference to the forgoing, including to FIGS. 1A to 4, for context, FIGS. 5 to 7 illustrate several example methods 500, 600, and 700 of the present disclosure. As those skilled in the art will readily appreciate, each method 500, 600, and 700 may be performed using one or more components of a CT system of the present disclosure, including the components shown in and described relative to FIGS. 1A to 4 and 8. Those skilled in the art will readily appreciate that each of methods 500, 600, and 700 do not necessarily describe all of the functionality of each of the relevant components. Rather, each highlights one or more particular aspects for the sake of illustration. However, those skilled in the art will readily understand, using this disclosure as a guide, how to implement additional and/or alternative functionality(ies) disclosed without undue experimentation. While methods 500, 600, and 700 are not described with continual reference to any of FIGS. 1A to 4 and 8, those skilled in the art will also readily understand, by virtue of the use of common terminology throughout this disclosure, how aspects of the methods 500, 600, and 700 relate to all other relevant parts of this disclosure.

It is noted that the terms “first” and “second” as used in methods 500, 600, and 700 do not refer to ordinality, any relative importance, or relationship between differing elements modified by the same one of these words. Rather, “first” and “second” are merely used to indicate that the relevant method 500, 600, and 700 contains two of the same type of element and that, in the method, each of those elements has a particular role relative to the other like element. It is further noted that the number of particular elements addressed in each of the methods 500, 600, and 700 is not necessarily representative of the number of such elements that would be present in an actual implementation. On the contrary, the number of elements in each method 500, 600, and 700 is simply the minimum number of elements used to convey the relevant functionality(ies) to the reader.

Turning first to FIG. 5, the example method 500 of FIG. 5 includes a method of notifying a first person that they have been in transmissive contact with another person that has self-reported as having been infected by an infectious agent. In this example, the first person is carrying a mobile device, or “first mobile device”, that includes a first instance of CT software, and the second person also is carrying a mobile device, or “second mobile device”, that includes a second instance of the CT software. For the sake of this example, it is the first mobile device that performs method 500.

At block 505, a first CT key that the second mobile device transmits is received. As discussed above, when one mobile device (here, the first mobile device) receives a CT key from another mobile device (here, the second mobile device), this is an indication that the CT software has determined that the first and second persons are in transmissive contact or suspected transmissive contact with one another. Regarding suspected transmissive contact and as discussed above, in some embodiments the determination of whether transmissive contact has occurred is further refined using location metadata to determine whether or not two mobile devices, such as the first and second mobile devices in this example, are in the same separated space (e.g., in the same room or at the same workstation, etc.).

At block 510, an infected CT key is received. As discussed above, an infected CT key is a CT key associated with a person, here, the second person, self-reporting that they have been infected with an infectious agent for which the CT software has been deployed and/or parameterized. As noted above, the infected CT key may, in some deployments of a CT system, be pushed from a server in response to the second person self-reporting, while in some deployments the infected CT key may be pulled from the server. As discussed above, depending on a number of possible factors, such as when the first CT key was received relative to when the second person self-reported and how many CT keys the second mobile device generated from the time it generated the first CT key and the time the second person self-reported, at block 510 the first mobile device may receive more than one infected CT key.

At block 515, the infected CT key and the first CT key are matched to one another. As discussed above, this matching indicates that during a determined transmissive contact, i.e., the transmissive contact at which the first mobile device received the first CT key, the first and second mobile devices exchanged their then-current CT keys. Specifically, the second mobile device transmitted the first CT key (which is now equivalent to the infected key) to the first mobile device, and the first mobile device transmitted a second CT key to the second mobile device. In the context of FIGS. 1A, 2, 3, and 4, these first and second CT keys may be equated to the CT keys exchanged on Day 5.

At block 520, based on the matching at block 515, the first mobile device notifies the first person of the transmissive contact. This notification may be through any one or more HMI features on the first mobile device, such as an aural alert, a haptic alert, a message alert, or any combination thereof. As discussed above, this alert is anonymous in that it does not identify either the person that self-reported or when the transmissive contact occurs. A purpose of the notification may be to prompt the first person to get tested to determine whether or not they have been infected with the infectious agent. In an example, if the first person were to test positive for the infectious agent, they would be instructed to self-report so that their CT key(s), such as the second CT key mentioned above, could be stored on the server for pulling, or perhaps pushing, out of a server as an infected CT key, just like the infected CT keys that they received as discussed above.

As noted above, the determination of transmissive contact can be enhanced using location metadata. In the context of FIG. 5, in an example the method 500 may be made to consider location in the transmissive-contact determination by adding a number of additional blocks, such as blocks 525 through 545. At block 525, first location metadata identifying a location (e.g., particular separated space, such as a certain room or certain workstation, etc.) of the first mobile device at a time proximate to receiving the first CT key from the second mobile device is determined. In this example, “proximate” generally means within, at most, two seconds of the first and second mobile devices exchanging the first and second CT keys.

At block 530, second location metadata may be received from the second mobile device indicating the location of the second mobile device. The first mobile device may receive the second location metadata in conjunction with receiving the first CT key, meaning that the second mobile device transmits the second location metadata at or around (e.g., within a second) the same time it transmits the first CT key.

At block 535, the first and second location metadata are compared with one another to determine whether or not the locations of the first and second mobile devices are the same. At block 540, when the first and second location metadata match, the first mobile device stores the first CT key in its memory. Because the first mobile device has stored the first CT key, the first CT key is available for matching at block 515. At block 545, when the first and second location metadata do not match, the first mobile device does not store the first CT key. Therefore, the first CT key is not available for matching at block 515, so the first mobile device will not notify the first person that transmissive contact had occurred when their first mobile device exchanged the first and second CT keys with the second mobile device.

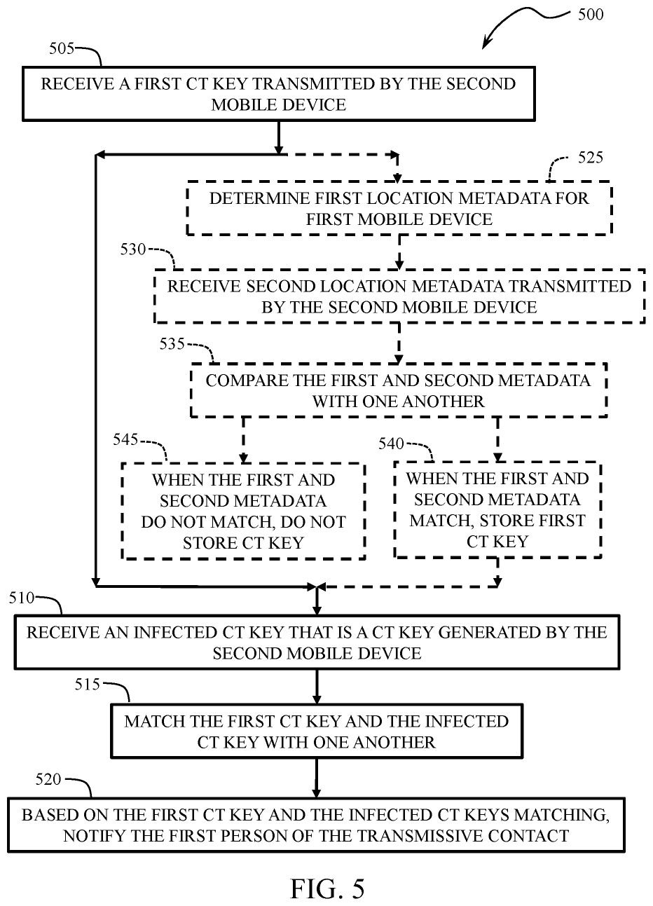

FIG. 6 illustrates a method 600 of controlling a mobile device deployed in a CT system of the present disclosure, such as any of the CT systems described herein and/or illustrated in the accompanying drawings. In this example and for context, the CT system of the method 600 includes at least first and second mobile devices and a server for facilitating certain functionality of the CT system. As with method 500, the first and second mobile devices are carried, respectively, by first and second persons and are presumed to be in immediate possession of their respective carriers when the CT system determines that a transmissive contact has occurred between the first and second mobile device and, therefrom, presumptively between the first and second persons. In this example, all of the functionalities of the method 600 are performed by the first mobile device. However, these same functionalities would also be performed by any other mobile device that is part of the CT system, such as the second mobile device.

At block 605, first-mobile-device CT keys are continually generated serially and stored. As discussed above, the continual generation of the first-mobile-device keys may proceed in any suitable manner, such as on a set time schedule (e.g., daily, hourly, every 10 minutes, etc.) or by triggering from one or more events (e.g., moving from one separated space to another, booting-up of the first mobile device, etc.) or any suitable combination thereof. Each of the generated first-mobile-device CT keys is stored in memory aboard the first mobile device. In embodiments in which the infectious agent has a well-defined incubation period, the CT software aboard the first mobile device may purge any stored first-mobile-device CT keys that are from a period prior to the presumed beginning of the incubation period. For example, if the incubation period is 9 days, then, as a current time, the CT software may purge any stored first-mobile-device CT key(s) older than 9 days. In another embodiment, incubation period consideration may not be handled as a purging but rather at the time that the first person self-reports that they have been infected—or likely infected—with the infectious agent. In such embodiments, the CT software aboard the first mobile device may only send the first-mobile-device CT keys stored during the 9 days leading up to the self-reporting. As those skilled in the art will readily appreciated in situations wherein the incubation period of the relevant infectious agent is well-established, limiting the number of the first-mobile-device CT keys stored and/or transmitted will minimize the number of transmissive contacts that are truly not transmission events.

At block 610, the first mobile device receives a self-reporting of (suspected) infection by the first person. As discussed elsewhere herein, this may be done by the CT software aboard the first mobile device displaying a self-reporting feature, such as a self-reporting soft control (e.g., button, slider, etc.) and then receiving a user selection or activation of the self-reporting feature. As discussed above, any self-reporting may or may not be accompanied by the first person providing any additional information, such as a phone number and/or other contact information, for example, if a CT team wants/needs to follow up with the first person regarding the self-reporting. Also noted above, the self-reporting may be predicated on the occurrence of any one or more events, such as receiving test results indicating infection, receiving a professional assessment indicating infection, or making a self-diagnosis of infection based on personal assessment of experienced symptoms, among others.

At block 615, based on receiving the self-reporting of infection at block 610, the first mobile device transmits a set of the stored first-mobile-device CT keys to the server. As discussed elsewhere herein, the CT system will use the first-mobile-device CT keys transmitted with the self-reporting to anonymously notify other persons that are part of the CT system, such as the second person, that the CT system has determined that they have been in transmissive contact with a person (here, anonymously the first person) that has been infected, or is at least suspected to have been infected, with the relevant infectious agent. In some embodiments, this is done by other mobile devices, such as the second mobile device pulling from the server the set of the first-mobile-device CT keys as a set of infected keys. The CT software on each mobile device in the CT system, such as the CT software aboard the second mobile device, then uses the infected CT keys to determine whether or not any of these infected CT keys matches any of the exchanged CT keys it has stored in its own memory based on prior transmissive contacts. As noted above relative to block 605, if the CT software aboard the first mobile device controls the number of the first-mobile-device CT keys transmitted based on a known incubation period, for example, either by memory purging or transmission control, the set of the first-mobile-device CT keys transmitted at block 615 may be limited to only the ones of the first-mobile-device CT keys from during the incubation period. Alternatively, the server may be configured to handle this optional functionality, for example, by only making available the ones of the first-mobile-device CT keys received from the first mobile device that are from the incubation period.

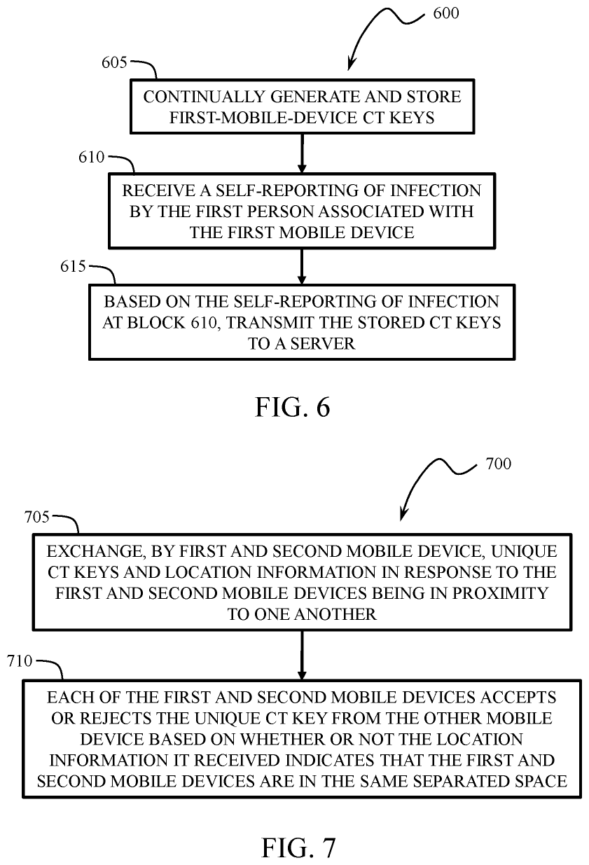

FIG. 7 illustrates an example method 700 to determine whether transmissive contact has occurred between two people carrying, respectively, the first and second mobile device. In this example, each of the first and second mobile devices contains CT software configured in accordance with aspects of the present disclosure.

At block 705, the first and second mobile devices exchange unique CT keys and location information in response to whether the first and second devices are in physical proximity to one another. In some embodiments, and as noted elsewhere herein, each of the first and second mobile devices may continually generates their own unique and anonymous CT keys on a schedule and/or based on the occurrence of certain events. Physical proximity can be determined using any suitable means, such as signal strength of radios aboard the first and second device, among other things. In some embodiments, the physical proximity can be defined by whether or not one, the other, or both of the first and second mobile devices detects when the other one of the first and second mobile devices is within an envelope, or bubble, having a radius equal to any maximum transmissive distance established for the infectious agent at issue. As noted above, physical proximity can, but need not, be augmented, for example, with a transmissive time, which is equal to the minimum amount of time that two people need to be within the maximum transmissive distance for the infectious agent to be presumed to be transmitted from one of the two people to the other of the two people.

The location information may be, for example, location metadata, such as a separated space identifier that identifies the specific separate space that each of the first and second mobile device determines itself to be located in. As noted elsewhere herein, examples of separated spaces include, but are not limited to, rooms within a facility and workstations within a common workspace, among others.

At block 710, each of the first and second mobile devices either accepts or rejects the CT key received from the other one of the first and second mobile devices based on whether or not the location information it received from the other one of the first and second mobile devices indicates that these two mobile devices are in the same separated space. Acceptance and rejection can be based on the act of storing or not storing the received CT key. That is, if it is determined that the first and second mobile devices are located in the same separated space, then each of the two mobile devices will store the received CT key. This will, for example, make it available in the event that the person associated with the one of the first and second mobile devices that transmitted the CT key self-reports being infected so that the CT system, primarily the CT software aboard the relevant one of the two mobile devices, can determine whether or not the associated person was involved in a transmissive contact. Conversely, if it is determined that the first and second mobile devices are not located in the same separated space, then each of the two mobile devices will simply not store the received CT key. Therefore, it will not be available in the event that the person associated with the one of the first and second mobile devices that transmitted the CT key self-reports being infected. This precludes any determination that the two people were in transmissive contact with one another, which is a local result since they were in fact in two different-separated spaces where it would be impossible for transmissive contact to occur.

FIG. 8 illustrates an example instantiation of a CT system 804 of the present disclosure. In this example, on the hardware side, the CT system 804 utilizes one or more servers (represented here by a single server 808 for convenience) and a plurality of mobile devices 812(1) to 812(N) (or 812(1)-(N), for short) that are carried by people (e.g., members of a specific organization or members of the public at large) and can communicate data with one another over a communications network 816. In this example, the server 808 runs CT-server software 808S that is designed and coded to perform a variety of functionalities that facilitate the CT scheme that is desired to be implemented, and each of the mobile devices 812(1)-(N) runs corresponding CT software 812S (only one instance illustrated in the first mobile device 812(1)) that is also designed and coded to perform a variety of functionalities that facilitate the implemented CT scheme.

In some embodiments, functionalities that the server 808 provides may include, but not be limited to: receiving, from the plurality of mobile devices 812(1)-(N) and over the communications network 816, self-reports (collectively, 808SR) of infection by the possessors of the mobile devices; receiving, from the plurality of mobile devices and over the communications network, infected CT keys (collectively, 808IK) from the mobile devices; providing a dashboard user interface (UI) 808UI for use by an optional CT team/person 820; releasing, based on continual pull polling by mobile devices that are part of the CT system 804, infected CT keys to the mobile devices using the communications network (or perhaps pushing out infected keys); and/or using incubation information to determine which infected CT keys to release to the mobile devices over the communications network; among other functionalities, or any suitable subset thereof. Those skilled in the art will readily understand how to configure and code the server CT software 808S to perform any one or more of these functionalities using the present disclosure as a functional specification of sorts. It should go without saying that the server 808 includes, among other things: one or more microprocessors 808MP for executing the CT-server software 808S and other software (not shown) for appropriately controlling the server; various types of hardware storage media (i.e., memory) 808M for storing or holding, permanently or temporarily dependent on the purpose of the storing/holding, all data, such as infected CT keys and any data corresponding to the self-reports, stored on the server and all software, including the server CT software, that runs on the server; and one or more communications ports 808CP for allowing the server to communicate with the communications network.

Those skilled in the art will readily understand that each microprocessor 808MP can be any suitable microprocessor, such as a general processing unit. Those skilled in the art will also readily understand that the memory 808M is a collective representation of all physical memory that is part of the server 808, including, but not limited to transient physical memory, such as cache memory that may be aboard the microprocessor(s) 808MP and certain types of RAM, among others, and long-term-storage memory, such as, but not limited to, solid-state-device drives (e.g., DRAM, flash memory, etc.), magnetic storage media (e.g., disks, bubble, tape, etc.), and optical storage media, among others. Fundamentally, there is no limitation on the types of memory that can be used as the memory 808M of the server 808. For the sake of clarity, it is specifically noted that the terms “hardware storage medium” and “hardware storage media” exclude transitory signals, such as radio-frequency signals, optical signals, ultrasonic signal, and any wire, fiber, or other signal conductor(s) (including space) that are provided for the purpose of conducting transitory signals. Further, those skilled in the art will readily understand that each communications port 808CP may be any suitable type of communications port, such as a Ethernet port or a wireless communications port, among many others.

A desired characteristic of the mobile devices 812(1)-(N) is that the people that are participating in the CT of the CT system 804 carry them, or otherwise keep them at least within arm's reach, generally throughout the entire period during which CT is desired to be monitored. Presently, smartphones are virtually ubiquitous devices that their users typically carry with them nearly constantly throughout the day, and so, smartphones are presently one type of mobile device for implementing as the mobile client devices 812(1)-(N) of the CT system 804. Indeed, current smartphones typically have instruments and features, such as WI-FI® radios, Bluetooth® radios, graphic-display-based UIs, global positioning systems (GPSs), among others, that a CT system of the present disclosure, such as CT system 804 of FIG. 8, can leverage, such that they are a natural target for use as the mobile devices 812(1)-(N). In such cases, all that typically needs to be done to make them components of the CT system 804 is to load them with mobile CT software 812S, such as a downloadable software app, that is designed and coded to provide the smartphones with the requisite functionality. That said, those skilled in the art will readily appreciate that any or all of the mobile devices 812(1)-(N) can be any other suitable device, such as a smartbadge (e.g., issued by an organization implementing the CT system 804 for monitoring CT among its members, a smartwatch, or other personal smart device, among others. The form(s) of the mobile devices 812(1)-(N) is/are generally not critical; rather, it is the functionality such devices provide that is more important. That said, whatever the nature of the mobile devices 812(1)-(N), they must be devices that the users are willing to keep with them at the necessary times.

FIG. 8 illustrates example components of any one of the mobile devices 812(1)-(N), with these components being shown only relative to mobile device 812(1) to avoid cluttering FIG. 8. It should be understood that the components shown in connection with mobile device 812(1) can likewise be present in each of the remaining mobile devices 812(2) to 812(N). The components of the mobile device 812(1) may include at least one microprocessor 812MP, memory 812M, one or more network interfaces, such as cellular network radio 812CR and/or a WI-FI® radio 812WR, an electronic display 812ED, and one or more instruments (collectively represented by instrument(s) 8121) that provide and/or assist with obtaining data relating to the location and/or movement of the mobile device, such as a GPS, a BLE radio, a magnetometer, an accelerometer, etc. Like the memory of the server 808M, the memory 812M may be of any suitable types, such as any two or more of the types mentioned above relative to the server memory. The memory 812M contains, among other things, the mobile CT software 812S, stored CT keys that the first mobile device 812(1) has generated, any stored CT keys received from one or more other ones of the mobile devices 812(2) to 812(N), any infected CT keys received from the server 808, information relating to self-reporting, including reporting history and/or contact information for the CT team/person 820, and any other software and data for controlling operation of the first mobile device and for running any other software/apps that may be present on the first mobile device, among other things.

In this example, the mobile CT software 812S is designed and coded so that when it is executed by the microprocessor(s) 812MP it performs a variety of functionalities that may include, but not be limited to: continually generating new unique anonymous CT keys; storing the generated CT keys in the memory 812M; determining whether or not the first mobile device 812(1) is in proximity to another one of the mobile devices 812(2) to 812(N) (e.g., using the BLE radio or other instrument 8121 aboard the first mobile device); tracking the amount of time that the first mobile device is in proximity to another of the mobile devices; receiving a CT key from another one of the mobile devices; accepting or rejecting the received CT key; determining position data for the location of the first mobile device (e.g., by triangulation (such as use of the BLE radio), GPS, etc.); determining location metadata (e.g., using a stored map and the location data); presenting a self-reporting feature to the person carrying the first mobile device; presenting a self-reporting UI to the person carrying the first mobile device; receiving a self-reporting from the person carrying the first mobile device; sending to the server 808 stored CT keys generated by the first mobile device; receiving one or more infected keys from the server; and alerting the person carrying the first mobile device of involvement in a transmissive contact incident; among others, or any suitable subset thereof. Examples of these and other functionalities that the CT software 812S may provide are described elsewhere in this disclosure.

For the sake of illustration, FIG. 8 shows the first and second mobile devices 812(1) and 812(2) being within transmissive distance Dr of one another, with the transmissive distance being equal to the radius R of each of the idealized transmissive envelopes, or transmissive bubble 812B(1), 812B(2), surrounding the corresponding mobile device and centered at the onboard antenna(s) (not shown) of the corresponding BLE radio. As noted above, each of the first and second mobile devices 812(1) and 812(2) can use signal strength of the received BLE signal to estimate the distance D_{A-A} from its BLE radio antenna to the BLE antenna of the other one of the first and second mobile devices. When each of the first and second mobile devices 812(1) and 812(2) recognizes that the inter-antenna distance D_{A-A} is less than the transmissive distance Dr, it determines that it is within transmissive proximity of the other one of the first and second mobile devices.

The communications network 816 can be any one or more communications networks/paths needed for the mobile devices 812(1)-(N) and the server 808 to communicate with one another. Examples of such networks/paths include, but are not limited to, cellular-service networks, the Internet, wide-area networks, and local area networks, among others, and any interconnections therebetween and to server 808 and the mobile devices 812(1)-(N).

As an example use case, and referring to FIGS. 1A through 8 for context and relevant components, features and aspects of CT methodologies and CT systems that can be deployed in this use case, low-energy beacons, such as BLE beacons, may be used in a cleanroom scenario to provide indoor positioning for employees located in the cleanroom. The mobile device of an employee that works in the cleanroom and is in proximity (e.g., within 6 feet, 8 feet, etc.) of one or more other employees located just outside of the cleanroom and each carrying a mobile device will not exchange CT keys with the mobile device(s) of the employee(s) just outside the clean room, because the respective CT software aboard the involved mobile devices determine that the location metadata (e.g., separated-space identifiers) based on the beacons in the differing rooms are different. Of course, many other use cases are possible, as those skilled in the art will recognize.

As another example use case, and also referring to FIGS. 1A through 8 for context and relevant components, features and aspects of CT methodologies and CT systems that can be deployed in this use case, low-energy beacons, such as BLE beacons, may be used in a manufacturing facility that has a large open space occupied by multiple workers. In an example scenario, Emily and Eric may have their own workstations within the open space, and their workstations may be spaced apart by a greater distance than the maximum distance, such as six feet, that is a criterion for establishing transmissive contact. Initially, Emily and Eric are at their respective workstations with their mobile devices that each contain CT software of the present disclosure. Since they are not within six feet of one another at this point, their respective CT software does not attempt to exchange CT keys as described above. While Emily and Eric are at their workstations, their CT software determine the locations of the respective mobile devices using the low-energy beacons to triangulate their positions. For example, their workstations may be mapped into a map (e.g., floorplan) of the facility and the map stored as map data aboard the mobile devices, such that the location metadata, e.g., separated-space identifiers, may be, for example, “Emily's Workstation” and “Eric's Workstation”, respectively. It is noted that while these two workstations are in the same physical, non-partitioned room, they may be characterized in the CT system as separate spaces because they are simply too far apart from one another for an infection to spread to people at those workstations, here, Emily and Eric.

In an extension of this scenario, Eric leaves his workstation, along with his mobile device, to meet with Emily in her workstation. When Eric's mobile device is within six feet of Emily's mobile device, their respective CT software begins the process of exchanging keys and location metadata. Because Eric's mobile device is continually updating location information by triangulating using the low-energy beacons in the manufacturing facility, his CT software updates his location metadata to “Emily's Workstation”, such that when Emily's and Eric's mobile devices exchange keys and check each other's location metadata, the location metadata accompanying both CT keys, here, “Emily's Workstation”, matches one another. This causes each of the mobile devices to store the received CT key, signifying that transmissive contact has occurred between Emily and Eric for the purposes of contact tracing.

In each of the examples above that use mobile-device location, the local positioning system may use beacons for triangulation. For example, BLE-beacon-based indoor positioning systems are known, as are techniques for triangulating position using BLE beacons. However, for the sake of illustration some aspects and example implementations are described briefly here. Generally, the BLE beacons are located at fixed known positions relative to a facility, such as inside one or more spaces inside the facility, outside the facility, and/or surrounding the facility. When the BLE radio receiver of a mobile device picks up a signal from each of a number, N, of beacons, a suitable triangulation algorithm that uses relative signal strength can triangulate the position of the BLE radio receiver within or relative to the facility. In some embodiments, the positioning calculation can be performed every second or so such that the position of the corresponding mobile device can essentially be determined in real time.

Signal strength of signals from at least 3 BLE beacons is used to triangulate position. Example criteria may be that: 1) at least one BLE signal shall be measured by each mobile device with a level of −70 dBm or stronger; 2) at least 3 BLE signals shall be measured by each mobile device with a level of −80 dBm or stronger, including the dominant beacon measured at −70 dBm or stronger; and 3) at least 6 BLE signals shall be measured by each mobile device (including the dominant beacon at −70 dBm or stronger, plus beacons at −80 dBm or stronger, plus other beacon(s) detected in the area.

The output of the location algorithm may be a 3D location fix plus a confidence circle typically corresponding to 90% confidence, as well as geonotifications, for example, ID, location name, content, and end time. The location algorithm may, for example, include a foreground mode (BLE/GPS/map) and a background mode, support indoor/outdoor transitions, be device-centric, and able to work offline. Geonotification may include a beacon proximity mode and have a fine mode that leverages background location.

It is noted that while BLE signals are used in specific examples throughout this disclosure for the various proximity, location, and communication functionalities, other signals, including other radio signals, may be used. In addition, different signal types, such as radio signals of differing types (e.g., WI-FI®, Zigbee®, etc.), optical signals, and/or ultrasonic signals, may be used for differing functionalities. For example, the signal type used for proximity may be different from the signal type used for location. As another example, the signal type for communications may be different from one or both of the signal type(s) of the proximity and location functionalities.

Each mobile device may be any mobile computing device, such as a smartphone, smartwatch, smartbadge, etc., that has the requisite processing power, memory, and communications device(s) (e.g., radio(s)) to perform the necessary functions. Those skilled in the art will readily understand the hardware requirements needed for a mobile device to implement a CT system of the present disclosure. Those skilled in the art will also readily understand how to configure the software necessary to perform the functionalities needed for such mobile devices. Similarly, those skilled in the art will readily understand the hardware needed for the server(s) described herein, as well as the communications networks (e.g., cellular, Internet, local area, wide area, personal area, etc.) needed to enable the functionalities described herein. In addition, those skilled in the art will readily understand how to configure a location positioning system of the present disclosure, such as a BLE-beacon-based location positioning system.

Regarding software for the mobile devices and the server for implementing a CT system and/or a CT methodology of the present disclosure, those skilled in the art will readily understand how to create the software modules, app(s), algorithms, etc., and/or other code, necessary to cause the relevant hardware to perform the necessary functions. Consequently, the software need not be described in detail for those skilled in the art to implement the broad features, singly or in any suitable combination, of the present disclosure without undue experimentation.

It is noted that the foregoing examples describe the people carrying the mobile devices as “organization members”. However, the people do not necessarily need to be members of an organization. For example, at least one of the people may be of another type, such as a visitor to a monitored facility, a temporary contractor working at the monitored facility, or another type of non-member, such as any member of the general public in a geographic region, city, state, territory, nation, continent, island, etc., where aspects of any CT methodology and/or CT system of the present disclosure may be implemented.

The server-side functionalities of a CT system of the present disclosure may be implemented as a standalone CT monitoring software application or as part of other software, such as a critical event management software system. In some embodiments, the server-side functionalities may be part of a cloud-based multi-tenant system or it may be implemented on an onsite enterprise server. Fundamentally, there are no limitations on the manner and/or architecture in which the server-side functionalities may be implemented.

Various modifications and additions can be made without departing from the spirit and scope of this disclosure. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present disclosure. Additionally, although particular methods herein may be illustrated and/or described as being performed in a specific order, the ordering is highly variable within ordinary skill to achieve aspects of the present disclosure. Accordingly, this description is meant to be taken only by way of example, and not to otherwise limit the scope of this disclosure.

Exemplary embodiments have been disclosed above and illustrated in the accompanying drawings. It will be understood by those skilled in the art that various changes, omissions and additions may be made to that which is specifically disclosed herein without departing from the spirit and scope of the present disclosure.

The status of the claims of the current application stands as follows:

1. A method of notifying a first person that the first person has been in transmissive contact with a second person that has been infected with an infectious agent, wherein each of the first and second persons carries, respectively, a first mobile device and a second mobile device each containing contact-tracing (CT) software, the method being performed by the first mobile device and comprising:
receiving a first CT key transmitted by the second mobile device;
receiving an infected CT key indicating that the second person has been infected by the infectious agent;
matching the infected CT key and the first CT key to one another; and
based on the matching, causing the first mobile device to notify the first person of the transmissive contact.

2. The method of claim 1, further comprising:
determining first location metadata identifying a location of the first mobile device at a time proximate to receiving the first CT key;
receiving second location metadata transmitted by the second mobile device in conjunction with the second mobile device transmitting of the first CT key, the second location metadata identifying a location of the second mobile device;
comparing the first location metadata and the second location metadata with one another;
when the first and second location metadata match one another, storing the first CT key on the first mobile device; and
when the first and second location metadata do not match one another, not storing the first CT key on the first mobile device.

3. The method of claim 2, wherein determining the first location metadata includes triangulating, by the first mobile device, a position of the first mobile device using a plurality of wireless beacons.

4. The method of claim 3, wherein determining the first location metadata includes using the position and map data to determine the first location metadata.

5. The method of claim 4, wherein each of the first and second location metadata comprise a separated-space identifier.

6. The method of claim 4, wherein each of the first and second location metadata comprise a workspace identifier.

7. The method of claim 1, further comprising transmitting a second CT key to the second mobile device.

8. The method of claim 2, further comprising determining that the first and second mobile devices are within transmissive proximity of one another, wherein the transmitting of the second CT key to the second mobile device is based at least in part on the first mobile device determining that the first and second mobile devices are in transmissive proximity to one another.

9. The method of claim 8, further comprising:
monitoring an amount of time that the first and second mobile devices are within transmissive proximity of one another; and
comparing the amount of time to a transmissive threshold time;
wherein the transmitting of the second CT key to the second mobile device is based in part on the amount of time being equal to or greater than the transmissive threshold time.

10. The method of claim 7, further comprising transmitting, in conjunction with transmitting the second CT key, a location identifier indicating a current location of the first mobile device.

11. The method of claim 10, further comprising determining the location metadata by at least triangulating a position of the first mobile device using a plurality of wireless beacons.

12. The method of claim 11, wherein determining the location metadata includes using the position and map data to determine the location metadata.

13. The method of claim 12, wherein the location metadata comprise a separated-space identifier.

14. The method of claim 12, wherein location metadata comprise a workspace identifier.

15. A method of controlling a first mobile device deployed in a contact-tracing (CT) system that employs a server for facilitating the CT system and that includes a second mobile device, wherein the first mobile device is carried by a first person and the second device is carried by a second person, the method being performed by the first mobile device and comprising:
continually generating, serially, first-mobile-device CT keys and storing the first-mobile-device CT keys;
receiving from the first person a self-reporting of infection by an infectious agent; and
based on the receiving of the self-reporting, transmitting a set of the first-mobile-device CT keys to the server.

16. The method of claim 15, further comprising, in conjunction with the transmitting of the set of the first-mobile-device CT keys, transmitting contact information of the first person to the server, wherein the contact information allows a CT team to contact the first person regarding the self-reporting.

17. The method of claim 16, wherein receiving the self-reporting includes receiving a user-selection of a self-reporting feature of the first mobile device from the first person.

18. The method of claim 16, further comprising receiving the contact information from the first person.

19. The method of claim 15, further comprising:
determining that the first mobile device and the second mobile device are at least within a predetermined transmissive proximity of one another so as to assume that a transmissive contact has occurred between the first person and the second person;
based on the determining that the first and second mobile devices are at least within the predetermined transmissive proximity, transmitting a most recent one of the CT keys.

20. The method of claim 19, further comprising receiving and storing a second-mobile-device CT key from the second mobile device when the first mobile device has determined that the transmissive contact has occurred between the first and second persons.

21. The method of claim 20, further comprising:
in conjunction with receiving a second-mobile-device CT key, receiving second-mobile-device location metadata from the second mobile device;
determining first-mobile-device metadata;
comparing the second-mobile-device location metadata with the first-mobile-device metadata; and
storing the second-mobile-device key only when the second-mobile-device location metadata and the first-mobile-device metadata match one another.

22. The method of claim 21, wherein determining the first-mobile-device location metadata includes triangulating a position of the first mobile device using a plurality of wireless beacons.

23. The method of claim 22, wherein determining the first-mobile-device location metadata includes using the position and map data to determine the first-mobile-device location metadata.

24. The method of claim 23, wherein each of the first-mobile-device and second-mobile-device location metadata comprise a separated-space identifier.

25. The method of claim 23, wherein each of the first-mobile-device and second-mobile-device location metadata comprise a workspace identifier.

26. A method of determining whether transmissive contact has occurred between two people carrying, respectively, first and second mobile devices, the method comprising:
exchanging, between the first and second mobile devices, unique contact-tracing (CT) keys and location information in response to detecting that the first and second mobile devices are within a predefined transmissive proximity to one another;
wherein each of the first and second mobile devices accepts or rejects the unique CT key received from the other of the first and second mobile devices based on whether or not the location information indicates that the first and second mobile devices are in a same separated space as one another.

27. The method of claim 26, further comprising, after rejecting the unique CT key because the first and second mobile devices are not in the same separated space, generating a new unique CT key that initiates determination of whether or not the first and second mobile devices are in proximity to one another.

28. The method of claim 27, wherein each mobile device determines its location information using a local positioning system.

29. The method of claim 28, wherein the local positioning system is a low-energy radio beacon-based positioning system.

30. A machine-readable storage medium containing machine-executable instructions for performing the method of claim 1.
